(19) United States
(12) Patent Application Publication
(10) Pub. No.: US 2012/0167898 A1
(43) Pub. Date: Jul. 5, 2012

(54) Patient Immobilization Device For Radiotherapy Treatment System And Methods

(75) Inventors: Jeffrey C. Buchsbaum, Bloomington, IN (US); R. Victor Simoneaux, JR., Ellettsville, IN (US); Mark R. Wolanski, Indianapolis, IN (US); Joseph Simmons, Bloomington, IN (US); Amy Sandefur, Mooresville, IN (US)

(73) Assignee: Indiana University Research And Technology Corporation, Bloomington, IN (US)

(21) Appl. No.: 13/338,786

(22) Filed: Dec. 28, 2011

Related U.S. Application Data
(60) Provisional application No. 61/427,949, filed on Dec. 29, 2010.

(51) Int. Cl. A61F 5/37; A61N 5/00

(52) U.S. Cl. 128/870; 600/1

(57) ABSTRACT

A patient immobilization device for radiotherapy includes a one-piece composite body having a core and a carbon fiber reinforced skin encapsulating the core. The composite body further includes a posterior surface configured for mounting the composite body upon a radiotherapy support table, and an anterior surface defining a cradle. Positioning a patient upon the immobilization device and supporting the patient with the cradle enables emitting therapeutic radiation towards a craniospinal treatment site of the patient from a posterior side of the immobilization device while the patient is supported in a supine position. The emitted radiation may include proton beam radiation.

This application claims the benefit of the filing date of U.S. Provisional Patent Application Ser. No. 61/427,949, filed Dec. 29, 2010.

### TECHNICAL FIELD

The present disclosure relates generally to radiotherapy treatment systems, and relates more particularly to emitting therapeutic radiation from a posterior side of an immobilization device supporting a patient in a supine position.

### BACKGROUND

A variety of different radiotherapy treatments are used for treating human patients. In general terms, radiation in the form of gamma rays or charged particles such as electrons, protons and ions, is applied to undesired tissue such as tumor tissue within a patient's body. Exposure of the undesired tissue to radiation causes cell death of targeted tissues in a well known manner. Various radiotherapy procedures have been used for decades, many with notable success. All radiotherapy techniques, however, typically expose some healthy tissue to ionizing radiation in addition to the targeted, undesired tissue.

Various strategies are known in the art which attempt to minimize exposure of healthy tissue to ionizing radiation. In certain photon radiotherapy techniques, for example, treatment mechanisms are designed to focus photon radiation where needed, such as by using multiple emission sources from different locations about a treatment site. Radiation from the multiple sources is directed to converge at a treatment site and is thus of relatively high net intensity only within body tissue intended to be treated. This general strategy can limit radiation exposure of body tissue outside the zone of convergence. Proton beam therapies have been found to be particularly advantageous in certain treatments due to the manner in which avoidance of damage to healthy tissue is achieved. Those skilled in the art will be familiar with the general principles whereby protons lose energy within relatively narrow ranges of travel distance from an emission source, allowing the treatment to be relatively tightly focused upon undesired tissue, at least for certain types of procedures. While photon, proton, and other radiotherapy techniques have seen widespread success, there remains room for improvement, particularly with regard to treating certain areas of the human body.

### SUMMARY

In one aspect, a patient immobilization device for a radiotherapy treatment system includes a one-piece composite body having a core, a carbon fiber reinforced skin encapsulating the core, and defining a longitudinal axis extending between a proximal body end and a distal body end. The one-piece composite body further includes a posterior surface configured for mounting the one-piece composite body upon a radiotherapy support table, an anterior surface, and an outer peripheral edge. The anterior surface defines a cradle located adjacent the distal body end for immobilizing a patient in a supine position during radiotherapy treatment, and the outer peripheral edge includes a taper narrowing in a distal direction about the cradle.

In another aspect, a radiotherapy treatment system includes a motorized actuator system, a support table coupled with the motorized actuator system and having an outer table perimeter, and a patient immobilization device. The patient immobilization device includes a one-piece composite body having a core, and a carbon fiber reinforced skin encapsulating the core. The one-piece composite body further includes a cradle for supporting a patient in a supine position, and is cantilevered to the support table such that the cradle is positioned outboard of the outer table perimeter.

In still another aspect, a method of craniospinal radiotherapy includes supporting a patient in a supine position upon an immobilization device cantilevered to a support table, and emitting therapeutic charged particle radiation from a posterior side of the immobilization device toward a craniospinal treatment site of the patient, during supporting the patient in a supine position.

In still another aspect, a method of preparing a radiotherapy treatment system for service includes positioning a patient immobilization device upon a support table of the radiotherapy treatment system, the patient immobilization device including a one-piece composite body having a core and a carbon fiber reinforced skin encapsulating the core. The method further includes cantilevering the patient immobilization device to the support table such that a cradle of the patient immobilization device juts outwardly of the support table, and orienting a posterior side of the patient immobilization device toward a direction of incidence of therapeutic radiation at least in part via cantilevering the patient immobilization device to the support table.

### BRIEF DESCRIPTION OF THE DRAWINGS

FIG. 1 is a perspective view of a patient immobilization device according to one embodiment;

FIG. 2 is a sectioned view taken along line 2-2 of FIG. 1;

FIG. 3 is a sectioned view taken along line 3-3 of FIG. 1;

FIG. 4 is a sectioned view taken along line 4-4 of FIG. 1;

FIG. 5 is a perspective view of a radiotherapy treatment system, according to one embodiment;

FIG. 6 is another perspective view of the patient immobilization device of FIG. 1; and

FIG. 7 is a top view of the patient immobilization device of FIG. 1.

### DETAILED DESCRIPTION

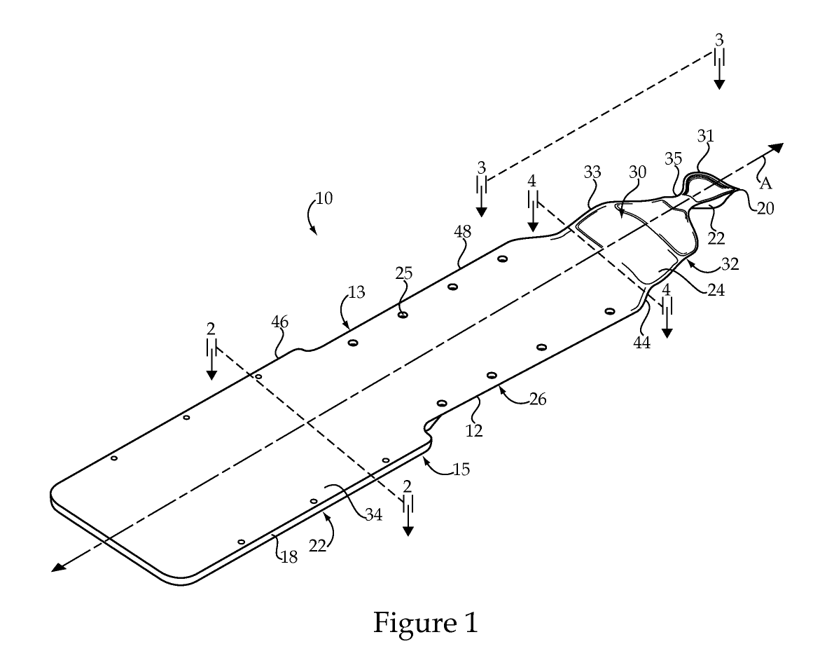

Referring to FIG. 1, there is shown a patient immobilization device 10 for a radiotherapy treatment system. Device 10 includes a one-piece composite body 12 defining a longitudinal axis A extending between a proximal body end 18 and a distal body end 20. Composite body 14 further includes a posterior surface 22 configured for mounting composite body 12 upon a radiotherapy support table as further described herein. A portion of posterior surface 22 which contacts a support table may be planar. Composite body 12 may further include a proximal body segment 46 which includes proximal body end 18, and a distal body segment 44 which includes distal body end 20. A middle body segment 48 extends between proximal and distal segments 46 and 44. Posterior surface 22 may be located on a posterior side 15 of composite body 12. An anterior surface 24 may be located on an anterior side 13 of composite body 12. Composite body 12 further includes an outer peripheral edge 26 extending about proximal, distal and middle body segments 46, 44 and 48.

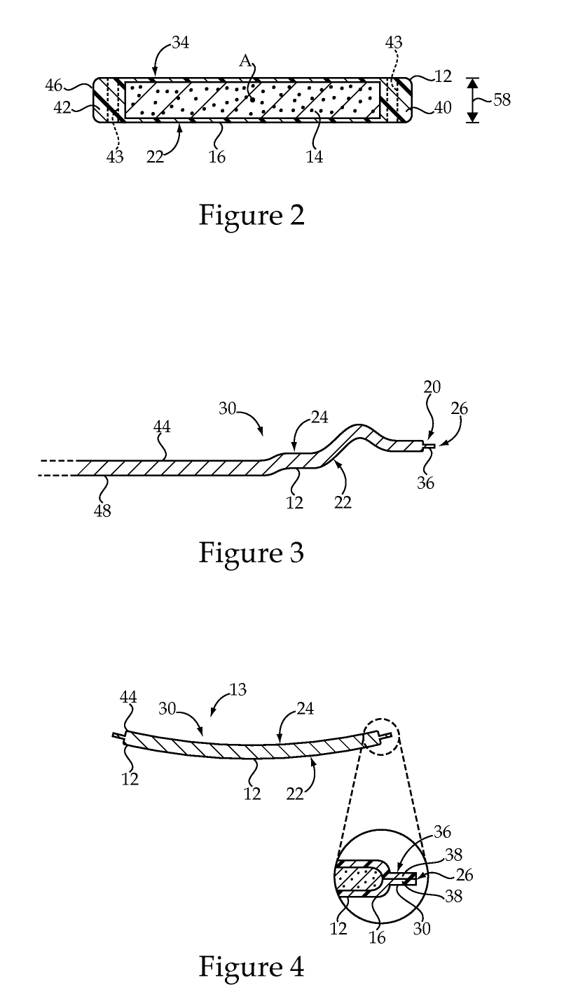

Referring also now to FIG. 2, there is shown a sectioned view along line 2-2 of FIG. 1. The view shown in FIG. 2 is through proximal segment 46, and illustrates a thickness 58 between surfaces 22 and 24. Thickness 58 may be uniform throughout proximal segment 46, and may be equal to about 5 cm or less, and in certain embodiments equal to about 4 cm. As used herein, the term “about” may be understood in the context of rounding to a consistent number of significant digits. Accordingly, “about” 4 cm means from 3.5 cm to 4.4 cm, and so on. Composite body 12 may further include a core 14 such as a homogeneous polymeric foam core, and a carbon fiber reinforced skin 16 encapsulating core 14. Skin 16 may include carbon fibers encased in a suitable resin. The fibers may be in a weave pattern, such as a plain weave, a non-woven pattern, or still another pattern, and may include any suitable orientation or combination of orientations such as fiber orientations transverse, parallel, or anti-parallel axis A. In the illustrated embodiment, immobilization device 10 includes a first support strut 40 and a second support strut 42 extending longitudinally within composite body 12 from proximal body end 18. Struts 40 and 42 may include carbon fiber reinforced support struts formed integrally with composite body 12, and may be located entirely within proximal body segment 46. Body 12 may be free from metallic materials, and consist essentially of skin 16, core 14 and struts 40 and 42, in one embodiment. The materials used in constructing device 10 are generally known in the art. A plurality of mounting apertures 43 may extend through support struts 40 and 42 and thus communicate between an anterior side 13 and a posterior side 15 of body 12, for reasons which will be further apparent from the following description.

Anterior surface 24 may include a non-planar surface defining a cradle 30 located adjacent distal body end 20 for immobilizing a patient in a supine position during radiotherapy treatment. A second anterior surface 34 is located upon proximal and middle segments 46 and 48, and adjoins surface 24. A more distal portion 31 of cradle 30 may have the form of a headrest, while a more proximal portion 33 of cradle 30 may be shaped generally as a negative image of an upper torso of a patient. A middle portion 35 of cradle 30 provides a patient neck support. Cradle 30 may smoothly transition to planar anterior surface 34. Outer peripheral edge 26 may include a taper 32 narrowing in a distal direction about cradle 30. In other words, a width of composite body 12 in a direction normal to axis A may decrease in a proximal to distal direction about cradle 30, such that on the average the width of body 12 becomes narrower in a distal direction. In the illustrated embodiment, taper 32 may be compound such that outer peripheral edge 26 includes a humanoid profile about cradle 30, and imparting a humanoid shape to distal segment 44. A taper formed by linear edges would not fairly be understood to have or form a humanoid profile, and would not typically provide the nozzle access advantages further discussed herein. By way of comparison, outer peripheral edge 26 may include a non-humanoid profile extending about anterior surface 34, and such that each of proximal and middle body segments 46 and 48 has a rectangular shape in at least certain embodiments. It may be noted that cradle 30 is shaped to “cradle” a head, neck and at least a portion of a torso of a patient. Less or more of a patient's body might be “cradled” as that term is intended to be understood herein. It should further be understood that “cradle” implies at least some capacity to restrict positioning of a patient's body in both a side to side direction and a proximal to distal direction. An immobilization device having the form of a flat panel or the like, for instance, would not likely be fairly said to include or define a cradle.

Referring also now to FIG. 3, showing a sectioned view taken along line 3-3 of FIG. 1. It may be noted that cradle 30 includes an undulate longitudinal profile, and a non-uniform thickness decreasing in a distal direction toward distal end 20. The non-uniform thickness may be less than thickness 58. The undulate longitudinal profile facilitates supporting and immobilizing a supine patient in cradle 30, in other words placing some restriction on patient position in a proximal to distal direction. It may also be noted that the undulate longitudinal profile corresponds generally to what might be described as a humanoid longitudinal profile, not to be confused with the humanoid edge profile of outer peripheral edge 26. It may also be noted from FIG. 3 that outer peripheral edge 26 may be located on a projecting flange 36. Flange 36 may reside on distal and middle segments 46 and 48. A bite block support or other instruments may be mounted to flange 36 in certain embodiments. Insertion points for bite block supports or other instruments, in the form of molded-in apertures, cutouts or the like might also be formed in body 12, and in particular in flange 36. Holes 25 may be formed in middle segment 48, extending all the way or only partly through body 12, for attaching patient-securing straps or the like, in certain embodiments.

Referring also now to FIG. 4, showing a sectioned view taken along line 4-4 of FIG. 1, it may further be noted that cradle 30 may include a latitudinal profile which is concave, as considered from anterior side 13. FIG. 4 also includes a detailed enlargement of a portion of composite body 12 in which it is readily apparent that projecting flange 36 may include abutting layers 38 of carbon fiber reinforced skin 16. The absence of any core material between layers 38 facilitates machined or molded-in mounting features for instruments and the like, as noted above.

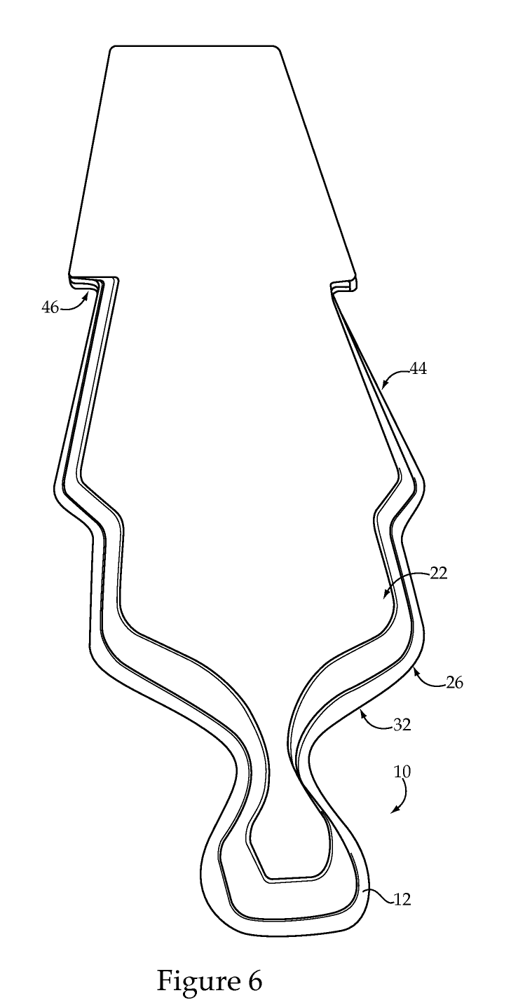

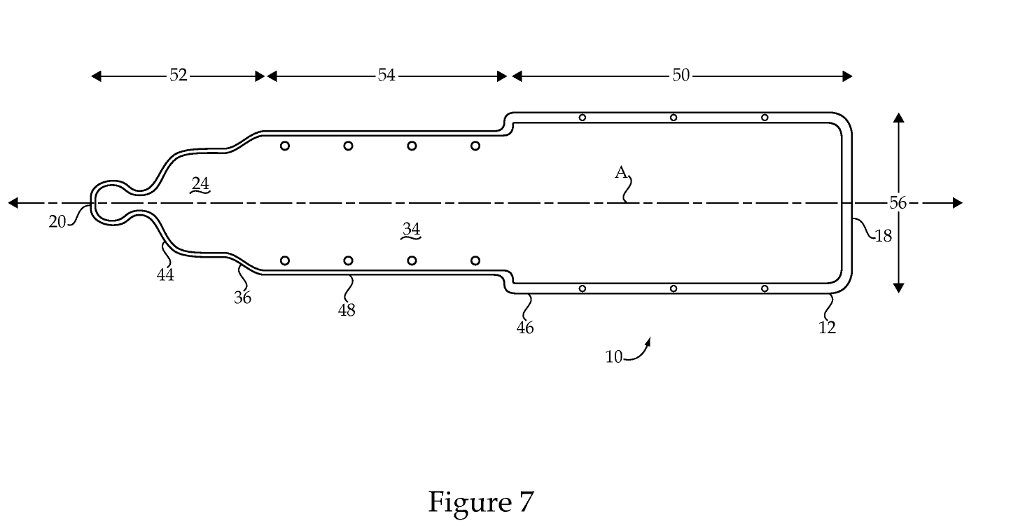

Referring to FIG. 6, there is shown a top view of immobilization device 10, illustrating further geometric and dimensional attributes. As noted above, distal segment 44 may have a humanoid shape, and each of middle and proximal segments 48 and 46 may have a rectangular shape. In view of the present description, it will readily understood that a rectangular shape is not a humanoid shape. It should be further understood that a shape which does not have an apparent head, neck and torso portion would also not be understood to have a humanoid shape. It may also be noted from FIG. 6 that distal body segment 44 includes a first axial length 52, middle body segment 48 includes a second axial length 54, and proximal body segment 46 includes a third axial length 50. In the illustrated embodiment, axial length 50 is large, axial length 54 is medium, and axial length 52 is small, relative to one another. Proximal body segment 46 may also have a width 56, which may be approximately equal to a width of a support table to which device 10 is to be mounted, although the present disclosure is not strictly limited as such. In a practical implementation strategy, a sum of axial lengths 50, 52 and 54 is equal to three times width 56, or greater. The sum of the axial lengths may be from about 175 cm to about 225 cm, and width 56 may be from about 50 cm to about 70 cm.

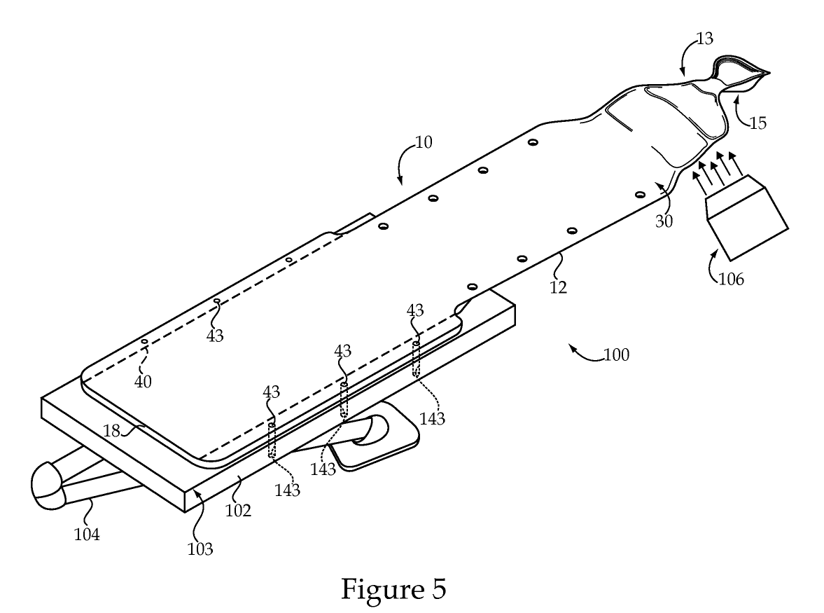

Turning now to FIG. 5, there is shown a radiotherapy treatment system 100 suitable for use with immobilization device 10. System 100 may include a motorized actuator system 104 such as a conventional robotic arm of the type commonly used in radiotherapy gantries. Actuator system 104 may be configured to adjust a radiotherapy support table 102 according to all possible degrees of freedom. In other words, actuator system 104 can raise or lower table 102, move table 102 back and forth or side to side horizontally, and may further be configured to rotate, pitch, or roll table 102. Table 102 may be coupled with actuator system 104, and includes an outer table perimeter 103. Immobilization device 10 is shown cantilevered to support table 102 such that cradle 30 juts outwardly of outer table perimeter 103. In one embodiment, device 10 may be positionable upon table 102 such that the entire spine of a patient, down to the iliac joints, is supported outboard of table perimeter 103. In a practical implementation strategy, about 60% or greater of a total axial length of body 12 may be located outboard of perimeter 103, and in certain instances about 65% or greater. A nozzle 106 such as a charged particle beam nozzle, or another type of therapeutic radiation emitting nozzle, is also shown in FIG. 5 and positioned such that therapeutic radiation may be emitted from a posterior side of immobilization device 10 toward a craniospinal treatment site of a patient supported in a supine position upon immobilization device 10. Cantilevering device 10 as described herein enables positioning nozzle 106 relative to a supine patient in a manner not possible, or at least impractical, with earlier patient support strategies, such as vertically below and directly underneath device 10. In other words, posterior side 15 may be oriented toward a direction of incidence of the charged particle radiation, such as protons, emitted from nozzle 106 once system 100 is prepared for service and a patient positioned upon device 10 as described herein.

Also shown in FIG. 5 are support struts 40 and 42 within composite body 12. It will be recalled that a plurality of apertures 43 may be formed in composite body 12 and extend through support struts 40 and 42, for example. Those skilled in the art will be familiar with the general concept of indexed devices and the like mounted to a radiotherapy support table. To this end, apertures 43 may be understood as indexing apertures arranged in a geometric pattern. Support table 102 may include another plurality of indexing apertures 143 also arranged in a geometric pattern. The geometric pattern of indexing apertures 43 may be congruent with the geometric pattern of indexing apertures 143. Embodiments are contemplated wherein the indexing apertures formed in patient immobilization device 10 are arranged in a geometric pattern identical to a geometric pattern of indexing apertures formed in support table 103. Another way to understand the principle of congruity between the respective sets of indexing apertures, is that immobilization device 10 may include at least two treatment positions upon support table 102 at which a plurality of indexing apertures 43 register with a plurality of indexing apertures 143. In FIG. 5, it may be noted that a total of six of indexing apertures 43 may be aligned with a total of six of indexing apertures 143, three of apertures 143 being shown, and an opposite three being hidden from view. It may further be noted that immobilization device 10 might be moved down and to the left in FIG. 5 to align a total of four of the respective sets of indexing apertures, or moved up and to the right to also align a total of four of the respective sets of indexing apertures. The number and configuration of indexing apertures 143 and 43 illustrated and described herein might be varied substantially without departing from the scope of the present disclosure. For instance, while apertures 43 and 143 are shown as bores through composite body 12 and support table 102, respectively, they might instead be formed as cutouts extending inward from peripheral edge 26 of composite body 12 and outer perimeter 103 of table 102. In any event, with indexing apertures 43 and 143 aligned as desired, fasteners, clamps, or still another coupling mechanism may be used to secure composite body 12 to support table 102 such that cradle 30 juts outwardly of support table 102, and in the cantilevered manner shown. As mentioned above, positioning immobilization device 10 in this general manner enables nozzle 106 to be oriented such that therapeutic radiation may be emitted toward a craniospinal treatment site of a supine patient supported within cradle 30, and from a posterior side of body 12 and thus a posterior side of the body of the patient. Radiation therefore does not pass, or is limited in passing, through an anterior side of the patient's body.

Referring now to FIG. 6, there is shown a bottom view of immobilization device 10, and illustrating posterior surface 22. By way of comparison, a planar posterior surface would not fairly be considered congruent with a non-planar anterior surface defining a cradle. It may be noted that posterior surface 22 includes a three-dimensional shape generally congruent with a shape of anterior surface 24. The humanoid profile of outer peripheral edge 26 within at least a portion of distal body segment 44 is also readily apparent in FIG. 6, as is its non-humanoid profile about proximal body segment 46.

### INDUSTRIAL APPLICABILITY

Referring to the drawings generally, those skilled in the art will be familiar with the general techniques employed to support patients for radiotherapy treatment upon a support table similar to support table 102. In preparing a radiotherapy treatment system for service such as system 100 described herein, steps may be undertaken similar to those used in connection with conventional radiotherapy practices, but with certain differences. Immobilization device 10 may be positioned upon support table 102 in the general manner described herein, and clamps, fasteners or the like used to releasably secure and position immobilization device 10 at a desired indexed location. The indexed location will typically predetermined based upon an indexed location previously used in imaging the treatment site within the patient, for reasons which will be apparent to those skilled in the art. This general positioning and coupling strategy enables immobilization device 10 to be supported in a cantilevered fashion upon support table 102 such that cradle 30 juts outwardly of support table 102. Posterior side 15 of composite body 12 may thus be positioned such that a space is defined between posterior side 15 and the floor of the treatment gantry. With cradle 30 jutting outwardly of support table 102, nozzle 106 can be positioned such that a path of a beam of radiation emitted from nozzle 106 does not need to pass through or about obstructions prior to entering composite body 10 and thenceforth interacting with targeted tissues within the supine patient.

Those skilled in the art will be familiar with the diversity of commercially available patient support and positioning systems available for radiotherapy treatment, and being tailored for various different purposes. Some systems enable adjustments in length or inclination, for example, or have features directed to stable and predictable or repeatable mounting. Other systems have mechanisms compensating for movement or misalignment of the hardware. While certain known strategies have worked well, and new ones are continually proposed, there remains ample room for improvement, particularly with regard to patient support and positioning systems used in charged particle radiotherapy, and especially proton therapy. In contrast to many types of photon therapy, uncertainty in the beam range of charged particles can be difficult to compensate. This is due at least in part to the greater sensitivity of protons and other charged particles to interaction with materials of the support system, especially changes in thickness and profile, potentially impacting path length. For these reasons, support and positioning systems well suited to photon radiotherapy are often inapplicable or inferior in charged particle techniques.

The present disclosure addresses these and other concerns at least in part by providing an immobilization device particularly well suited to charged particle radiotherapy that reduces uncertainty and day to day variation, in particular simplifying range calculations and dosimetry. Devices contemplated herein may be free of metal materials, as noted above, and also shaped to smoothly transition from one part of the device to another. Sharp transitions in thickness and profile are nonexistent, or virtually so, and what changes exist are gradual, such as the smoothly attenuated thickness of body 12 toward distal end 20. The shapes of the various parts of the immobilization device are also tailored to reduce the amount of material through which the charged particle beam needs to pass, while still providing for support and position control of the patient. The geometric and dimensional attributes of immobilization devices contemplated herein also enable better access by a large and unwieldy beam nozzle than that possible with many earlier designs. The ability to position the beam nozzle for posterior treatment of a supine patient is one example. Further advantages result from the humanoid shape of parts of the immobilization device, enabling better access with a beam nozzle, and less material to pass through, for head or neck treatments than that possible with known systems. These various advantages, and still others, are enabled without sacrificing strength, rigidity, or portability of the immobilization device. In certain embodiments, an immobilization device according to the present disclosure is configured to support patients up to about seven feet tall, and weighing up to about 325 pounds. According to one practical embodiment, when cantilevered to a support table as described herein, with about 60% or more of the axial length of the immobilization device outboard of the support table, 250 pounds can be rested upon distal segment 44 without inducing flexion sufficient to introduce uncertainties into dosimetry, or otherwise require compensation.

With conventional radiotherapy procedures, patients were often positioned in a prone orientation if radiotherapy was to be administered from a posterior side of the patient. Supporting patients in a prone orientation can not only be uncomfortable, but in some instances creates a risk of medically significant complications, particularly in relation to anesthesia and notably in pediatric treatments. Clinicians were thus previously faced with a choice between performing an often uncomfortable and potentially risky procedure versus foregoing a particular treatment strategy altogether. By providing for radiotherapy treatment of supine patients as set forth herein, these problems are expected to be reduced or eliminated altogether.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims.

What is claimed is:

1. A patient immobilization device for a radiotherapy treatment system comprising:
a one-piece composite body including a core, a carbon fiber reinforced skin encapsulating the core, and defining a longitudinal axis extending between a proximal body end and a distal body end;
the one-piece composite body further including a posterior surface configured for mounting the one-piece composite body upon a radiotherapy support table, an anterior surface, and an outer peripheral edge;
the anterior surface defining a cradle located adjacent the distal body end for immobilizing a patient in a supine position during radiotherapy treatment, and the outer peripheral edge including a taper narrowing in a distal direction about the cradle.

2. The patient immobilization device of claim 1 wherein the cradle includes an undulate longitudinal profile, and the outer peripheral edge includes a humanoid profile about the cradle.

3. The patient immobilization device of claim 2 wherein the anterior surface is non-planar, and the composite body further includes a second anterior surface which is planar and adjoins the non-planar anterior surface, and wherein the outer peripheral edge includes a non-humanoid profile extending about the second anterior surface.

4. The patient immobilization device of claim 1 wherein the outer peripheral edge is located on a projecting flange including abutting layers of the carbon fiber reinforced skin.

5. The patient immobilization device of claim 1 further comprising a first and a second support strut extending longitudinally within the one-piece composite body from the proximal body end.

6. The patient immobilization device of claim 5 wherein the first and second support struts include carbon fiber reinforced support struts formed integrally with the composite body, wherein the core is formed of a homogeneous foam, and wherein the one-piece composite body defines a plurality of mounting apertures extending through the support struts and communicating between the anterior and posterior surfaces.

7. The patient immobilization device of claim 1 wherein the one-piece composite body further includes a proximal body segment having a uniform thickness between the anterior and posterior surfaces, and a distal body segment having a non-uniform thickness, the non-uniform thickness being less than the uniform thickness and decreasing in a distal direction toward the distal body end.

8. The patient immobilization device of claim 7 wherein the one-piece composite body further includes a middle body segment, and wherein each of the proximal and middle body segments includes a rectangular shape, and the distal body segment includes a humanoid shape.

9. The patient immobilization device of claim 8 wherein each of the proximal, distal, and middle body segments includes an axial length, and the proximal body segment includes a width, and wherein a sum of the axial lengths is equal to three times the width or greater.

10. The patient immobilization device of claim 9 wherein the axial length of the proximal body segment is large, the axial length of the middle body segment is medium, and the axial length of the distal body segment is small.

11. The patient immobilization device of claim 10 wherein the uniform thickness is equal to about 5 cm or less, and wherein the sum of the axial lengths is from about 175 cm to about 225 cm, and the width is from about 50 cm to about 70 cm.

12. A radiotherapy treatment system comprising:
a motorized actuator system;
a support table coupled with the motorized actuator system, and including an outer table perimeter; and
a patient immobilization device including a one-piece composite body having a core, and a carbon fiber reinforced skin encapsulating the core, the one-piece composite body further including a cradle for supporting a patient in a supine position, and being cantilevered to the support table such that the cradle is positioned outboard of the outer table perimeter.

13. The radiotherapy treatment system of claim 12 wherein the one-piece composite body defines a longitudinal axis, and includes an axial length extending between a proximal body end and a distal body end, and the one-piece composite body being cantilevered to the support table such that about 60% or greater of the axial length is positioned outboard of the outer table perimeter.

14. The radiotherapy treatment system of claim 12 wherein the one-piece composite body further includes an anterior surface defining the cradle and having a first shape, and a posterior surface contacting the support table and having a second shape which is congruent with the first shape.

15. The radiotherapy treatment system of claim 14 wherein the one-piece composite body further comprises a distal body segment which includes the cradle, a proximal body segment, and an outer peripheral edge, and wherein the outer peripheral edge includes a humanoid profile within the distal body segment, and a non-humanoid profile within the proximal body segment.

16. A method of craniospinal radiotherapy comprising the steps of:
supporting a patient in a supine position upon an immobilization device cantilevered to a support table; and
emitting therapeutic charged particle radiation from a posterior side of the immobilization device toward a craniospinal treatment site of the patient, during supporting the patient in a supine position.

17. The method of claim 16 wherein the immobilization device includes a one-piece composite body having a carbon fiber reinforced skin encapsulating a homogeneous foam core, and wherein the step of supporting further includes supporting the patient within a cradle of the immobilization device jutting outwardly of the support table.

18. The method of claim 17 wherein about 60% or greater of an axial length of the one-piece composite body is positioned outboard an outer perimeter of the support table, and wherein the step of emitting further includes emitting the charged particle radiation from a nozzle positioned vertically below the immobilization device.

19. A method of preparing a radiotherapy treatment system for service comprising the steps of:
positioning a patient immobilization device upon a support table of the radiotherapy treatment system, the patient immobilization device including a one-piece composite body having a core and a carbon fiber reinforced skin encapsulating the core; and
cantilevering the patient immobilization device to the support table such that a cradle of the patient immobilization device juts outwardly of the support table; and
orienting a posterior side of the patient immobilization device toward a direction of incidence of therapeutic radiation at least in part via the cantilevering step.

20. The method of claim 19 wherein the step of cantilevering further includes coupling the patient immobilization device to the support table such that about 60% or greater of an axial length of the one-piece composite body is positioned outboard of the support table, and wherein the step of orienting further includes orienting the posterior side of the patient immobilization device toward a direction of incidence of charged particle radiation.
